(19) United States
(12) Patent Application Publication
(10) Pub. No.: US 2008/0275536 A1
(43) Pub. Date: Nov. 6, 2008

(54) PREVENTION OF DISPLACEMENT OF PROSTHETIC DEVICES WITHIN ANEURYSMS

(76) Inventors: Christopher K. Zarins, Portola Valley, CA (US); Peyman Benharash, Mountain View, CA (US); Oscar J. Abilez, Mountain View, CA (US)

Correspondence Address: Bozicevic, Field & Francis LLP;Stanford University Office of Technology Licensing, 1900 University Avenue, Suite 200, East Palo Alto, CA, 94303, US

(21) Appl. No.: 12/112,852

(22) Filed: Apr. 30, 2008

Related U.S. Application Data
(60) Provisional application No. 60/927,045, filed on Apr. 30, 2007.

(51) Int. Cl. A61F 2/84; A61F 2/82

(52) U.S. Cl. 623/1.11; 623/1.15; 623/1.13; 623/1.18

(57) ABSTRACT

Devices and methods are provided for reducing the lateral displacement of an endovascular device, e.g. a stent graft, within an aneurysm sac. A stabilization system comprising one or more stabilizing elements is inserted within the aneurysm space between an implanted device and the vessel wall. Filling this space prevents changes in curvature of the implanted endovascular device, and prevents longitudinal displacement, thereby providing for improved long-term stability and durability of endovascular repair.

### BACKGROUND OF THE INVENTION

Serious vascular defect can result when an area of weakened vessel wall causes a bulge, or bubble, to protrude out in a radial direction from the vessel. Such aneurysms can occur at various positions within the vasculature. Abdominal aortic aneurysms most often develop in the relatively long segment of aorta between the renal arteries and the bifurcation of the aorta into the right and left iliac arteries. Abdominal aortic aneurysms progressively enlarge at variable and unpredictable rates, and as they do, the involved aneurysm wall becomes weaker and thinner, and eventually ruptures. Rupture is relatively uncommon in abdominal aortic aneurysms less than five centimeters maximum transverse diameter, but the risk increases with increasing size. Rupture of abdominal aortic aneurysms has been responsible for approximately 15,000 deaths per year in the United States.

Endovascular repair of aortic aneurysms has been shown to be effective in preventing rupture of abdominal and thoracic aortic aneurysms and has reduced morbidity and mortality compared to open surgical repair. Consequently, endovascular repair is now extended to many patients who were not considered to be candidates for aneurysm repair in the past. However, despite the clear benefits of endovascular surgery in the early peri-operative period, there are significant concerns regarding the long-term stability and durability of endovascular repair, largely due to device migration or displacement over time.

When an aneurysm is located very close to the bifurcation of a trunk lumen into two branch lumens, treatment becomes especially difficult. One reason for this difficulty is because neither the trunk lumen nor either of the branch lumens provides a sufficient portion of healthy, lumen wall on both sides of the defect to which a straight section of single lumen stent or stent-graft can be secured. The stent or stent-graft must span the bifurcation site and yet allow undisturbed flow through each of the branch and trunk lumens.

Stent-grafts, which include a graft layer either inside or outside of a stent structure, are particularly useful for the treatment of aneurysms. The stent-graft provides a graft layer to reestablish a flow lumen through the aneurysm as well as a stent structure to support the graft and to resist occlusion or restenosis. In conventional approaches to repair, a small incision is made in a groin to form a small opening in one of the iliac arteries. A guide wire is passed up through the iliac artery into the aorta and through the aneurysm. A fabric tube graft is placed over a metal mesh stent which is mounted over the balloon portion of a large angioplasty type catheter. The fabric graft is folded longitudinally so that the entire apparatus can be loaded into a flexible plastic sheath and passed into the lumen of the iliac artery and up into the aneurysm. Once the prosthesis is properly positioned, the sheath is withdrawn.

Existing endovascular stent-graft devices are subject to adverse events including device migration, type I and III endoleaks, stent fractures, fabric tears, and modular disconnections. The risk of adverse events and migration increases with time and with increased aortic, aneurysm and iliac angulation and tortuosity. Device movement can result in loss of device fixation proximally, distally, or at modular junctions leading to endoleaks, re-pressurization of the aneurysm sac and aneurysm rupture. Additionally, increased lateral angulation of the stent-graft can lead to pressure erosion and perforation of the fabric resulting in type III endoleaks.

In order to counteract the continuous downward displacement forces which are exerted on all aortic devices (both abdominal and thoracic) by the pulsatility of blood a variety of endograft structures and designs have been developed. These include a variety of stent/fabric modular systems and unibody devices with suprarenal and infrarenal fixation systems with or without penetrating hooks and barbs for proximal and distal device fixation in the normal proximal aorta and normal distal aorta or iliac arteries.

Proximal fixation may prevent leaks to the outside of the graft, (which could maintain high arterial pressure within the abdominal aortic aneurysm and lead to progressive enlargement and rupture). It was also hoped that proximal fixation could prevent distal migration and collapse of the soft plastic fabric tube graft by the force of arterial blood flow. Proximal fixation of the graft has been attempted with the underlying stent. After withdrawal of the delivery sheath, the balloon is distended to expand the stent so that the stent presses the outer wall of the graft against the inner wall of the neck of the aneurysm. An alternative method of fixation involves a ring of interconnected, fine-metal hooks or barbs pre-sewn into the upper end of the graft. The metal hooks or barbs are “fired” into the wall of the neck of the aneurysm by inflation of the catheter balloon. Yet another known method is a combination of a stent and hooks or barbs, in which the hooks or barbs are welded to the stent.

However, there are major unsolved problems relating to proximal fixation of endovascular grafts. While previous attempts to devise methods of preventing endograft migration have focused on preventing longitudinal slippage of the stent grafts or stent graft elements by increasing its resistance to longitudinal or axial displacement, none of the currently available fixation mechanisms have successfully eliminated the potential for problems of endograft migration over time. Indeed, patients treated with endovascular repair require close, long-term image based monitoring with CT scans/ultrasounds/MRI's at regular intervals for their entire remaining lifetime in order to detect device migration, endoleaks and aneurysm enlargement.

The issue of axial movement, or migration, becomes even more significant when considering new and future generations of stent-grafts with branch-vessel technology. Such stent-grafts exclude the aneurysm sac while maintaining flow to side-branch vessels such as the carotid, celiac, superior mesenteric and renal arteries. This may be accomplished by creating short side-branches from the main endograft body and securing them to the branch vessels. A small amount of displacement of the main body of the endografts in the axial or lateral directions, could lead to dislodgment or occlusion of the side-branches potentially leading to re-pressurization of the aneurysm sac and potential interruption of flow through the branching vessel with severe consequences.

Due to the very large market available for endografts, there is intense interest in finding new ways of stabilizing aortic stent grafts and eliminating the potential complications and need for secondary procedures over time.

### Publications.

Patent publications relating to aortic aneurysms include, inter alia, USRE38146 “Method and apparatus for bilateral intra-aortic bypass”; U.S. Pat. No. 5,578,072, “Aortic graft and apparatus for repairing an abdominal aortic aneurysm”; U.S. Pat. No. 5,522,880, “Method for repairing an abdominal aortic aneurysm”; U.S. Pat. No. 5,489,295, “Endovascular graft having bifurcation and apparatus and method for deploying the same”; U.S. Pat. No. 6,475,466, “Methods for treating endoleaks during endovascular repair of abdominal aortic aneurysms”; U.S. Pat. No. 6,409,756, “Endovascular aortic graft”; U.S. Pat. No. 6,767,359 “Prosthesis for the repair of thoracic or abdominal aortic aneurysms and method therefore”; U.S. Pat. No. 5,643,208, “Balloon device for use in repairing an abdominal aortic aneurysm”; U.S. Pat. No. 4,577,631, “Aneurysm repair apparatus and method”; U.S. Pat. No. 7,112,217, “Biluminal endovascular graft system”; U.S. Pat. No. 7,004,964, “Apparatus and method for deployment of an endoluminal device”; U.S. Pat. No. 6,814,748, “Intraluminal grafting system”; U.S. Pat. No. 6,303,100, “Methods for inhibiting the formation of potential endoleaks associated with endovascular repair of abdominal aortic aneurysms”; U.S. Pat. No. 5,681,346, “Expandable stent forming projecting barbs and method for deploying”; U.S. Pat. No. 5,207,695, “Aortic graft, implantation device, and method for repairing aortic aneurysm”; U.S. Pat. No. 4,313,231, “Vascular prosthesis”.

Patent publications also include U.S. Pat. No. 4,733,665; U.S. Pat. No. 4,739,762; U.S. Pat. No. 4,776,337; U.S. Pat. No. 5,102,417; U.S. Pat. No. 5,316,023; U.S. Pat. No. 5,342,348; U.S. Pat. No. 5,464,449; U.S. Pat. No. 5,522,880; U.S. Pat. No. 5,571,170; U.S. Pat. No. 5,571,171; U.S. Pat. No. 5,578,071; U.S. Pat. No. 5,578,072; U.S. Pat. No. 5,683,453; U.S. Pat. No. 5,735,893; U.S. Pat. No. 5,741,274; U.S. Pat. No. 5,800,520; U.S. Pat. No. 5,807,404; U.S. Pat. No. 5,810,872; U.S. Pat. No. 5,824,037; U.S. Pat. No. 5,827,321; U.S. Pat. No. 5,855,600; U.S. Pat. No. 5,931,867; U.S. Pat. No. 6,042,597; U.S. Pat. No. 6,083,257; U.S. Pat. No. 6,106,548; U.S. Pat. No. 6,117,165; U.S. Pat. No. 6,123,722; U.S. Pat. No. 6,152,957; U.S. Pat. No. 6,179,868; U.S. Pat. No. 6,193,747; U.S. Pat. No. 6,248,122; U.S. Pat. No. 6,267,783; U.S. Pat. No. 6,361,558; U.S. Pat. No. 6,491,719; U.S. Pat. No. 6,533,811; U.S. Pat. No. 6,645,237; U.S. Pat. No. 6,660,032; U.S. Pat. No. 6,921,414; U.S. Pat. No. 7,037,331.

### SUMMARY OF THE INVENTION

Devices and methods are provided for reducing the lateral displacement of an aortic endovascular device, e.g. a stent graft, within an aneurysm sac. It has been found that lateral displacement, which can comprise lateral movement of an implanted device within the aneurysm space, is related to longitudinal displacement of the endovascular device. In the methods of the invention, a stabilization system comprising one or more stabilizing elements is inserted within the aneurysm space between an implanted device and the vessel wall. Filling this space prevents changes in curvature of the implanted endovascular device, and prevents longitudinal displacement, thereby providing for improved long-term stability and durability of endovascular repair.

Stabilizing elements for use in the methods of the invention are biocompatible elements of a size appropriate for vascular use; are usually expandable to facilitate delivery through, for example a catheter based system; and may be modular or unitary. The stabilization system is usually delivered under X-ray, CT, biplane angiography, MR or ultrasound guidance, either to the site of an existing endovascular device, or in conjunction with the delivery of an endovascular device. In such procedures, three-dimensional imaging may be used to guide the stabilizing element(s) into place using directional deflectable catheter system. For example, the space may be filled with a contrast agent prior to implantation in order to visualize the spatial requirements of the individual aneurysmal space.

In some embodiments of the invention, the stabilizing element is typically not anchored or fixed to the endovascular device. In other embodiments of the invention, the stabilization element is fixated to the endovascular device. In either such embodiment, the stabilizing element may be used with any endovascular device, including devices that have been implanted prior to the stabilization procedure.

Specific stabilizing elements of interest include balloons, e.g. one or more detachable balloons, which may be filled with a biocompatible matrix, e.g. cyanoacrylate, polyurethane, etc., including, without limitation, a two component matrix that polymerizes or stabilizes upon mixing; and matrices that are stabilized by heating or cooling at the time of expansion. A balloon is typically delivered to the site in an unexpanded form, and may be expanded through delivery of the biocompatible matrix material in order to expand in the region of least resistance. In some embodiments multiple balloons are delivered. In other embodiments a segmented balloon is delivered. A balloon may be a zero pressure inflation balloon.

In another embodiment of the invention, the stabilizing element(s) are sponges; self-expanding fluid; foam; pro-coagulants; glue; or other filler material, including polymerizing matrices, e.g. a two component matrix that polymerizes or stabilizes upon mixing; and matrices that are stabilized by heating or cooling at the time of expansion which may be protected by baskets or balloons.

Specific stabilizing elements of interest also include expandable devices, such as side stents; expandable spheres; nitinol coils, etc. Such devices are delivered in an unexpanded state, and are deployed at the aneurysm site in order to fill the space outside of the primary endovascular device. As the aneurysm space can vary widely, in some embodiments of the invention, multiple small stabilizing elements are deployed in order to accommodate individual variation in size.

When positioned in the vessel, the stabilizing elements will fill sufficient space in the aneurysm to substantially prevent lateral movement of the endovascular device. For example, the one or more stabilizing elements may bridge the area between the outer surface of the endovascular device and the inner surface of the blood vessel at the largest lateral dimension. By contacting both surfaces and filling the space between, the stabilizing element supports the endovascular device and prevents it from movement and change in curvature. If desired, during or following deployment the position and/or movement of the endovascular device may be imaged in order to assess the positioning and contacts of the stabilization element(s).

In one aspect of the invention, a method is provided for the treatment of aneurysmal arterial disease, the method comprising implantation of an endovascular device; and implantation of one or more stabilization elements that fill the aneurysmal space and decrease lateral displacement of the endovascular device. The stabilization element(s) can be delivered over a wire from above the endovascular device, for example by brachial access site; or can be delivered parallel to the primary module or iliac modules after full deployment of the endograft.

In such a method, the stabilization elements are implanted at the time of implantation for the primary endovascular device. Such a method may include the steps of introducing a double catheter comprising a graft system of a primary endovascular device and one or more stabilizing element(s) to the vessel, advancing the graft system through at least one artery, e.g. via the contralateral iliac access sheath; positioning the first end of the graft system in the aorta on one side of the aneurysm, the second end of the graft system extending across the aneurysm; and deploying the first end of the graft system in the aneurysm in a manner that does not substantially occlude the aorta. The step of deploying may include expanding the aortic stent radially outwardly to secure the first end of the graft system in the aorta. The graft system may further include an iliac stent attached to the second end of the graft system; then the method further comprises deploying the iliac stent in the iliac artery. The one or more stabilizing element(s) may be deployed following deployment of the aortic stent. When an iliac stent is used, the one or more stabilizing element(s) may be deployed before or after deployment of the iliac stent.

In some embodiments of the methods, a stabilizing element is deployed following deployment of the endograft, where the element is then attached to the endograft by any convenient method, e.g. with glue, barbs, hooks, etc. Where a glue is used, the catheter may be modified to provide delivery of the glue. For example, a catheter may separately deliver each component of a two component glue, which is then mixed in situ. Where the stabilizing element is attached by an attachment element, for example a barb, hook, Velcro, etc., the attachment element may make use of existing sites on the endograft to create an attachment point.

In one aspect, a delivery device is provided that can deliver an endovascular device, which may comprise one or more stent grafts, e.g. an aortic graft and/or an iliac graft; and a stabilization system, which may comprise one or more stabilization element(s) as described here. The delivery device may comprise a double lumen catheter for each component of the system. In some embodiments, the delivery device comprises an iliac stent and a stabilization system. For example, the device can be delivered via a “double-lumen” sheath to deploy the iliac limbs, leaving a wire outside the aneurysm sac for use with the stabilization system.

In another aspect of the invention, a method is provided for the stabilization of an implanted endovascular device, where the device, e.g. an aortic stent graft, has been deployed in a procedure prior to deployment of the stabilization element(s). In such a method, a catheter comprising a stabilization system of one or more stabilizing element(s) is introduced to a vessel, advanced through at least one artery; and deployed to fill the aneurysmal space.

The methods and devices of the present invention provide a means of augmenting resistance to migration of aortic endografts. The three-dimensional position (curvature) of the endovascular device is maintained within the aneurysm sac by filling the space between the endograft and the aneurysm wall once the device is in place, or as it is being deployed. By preventing lateral displacement of the middle portion of the endograft over time, the proximal and distal ends of the device will remain in place relative to the proximal and distal neck regions of the aneurysm, i.e. the regions where the endovascular device is designed to apply fixation forces by a variety of mechanisms. This invention is usable with all existing and future stent-graft constructs and it is independent of them. The various embodiments of this invention rely on filling the space in the aneurysm sac between the endografts and the aneurysm wall, thus preventing the possibility of increase/decrease in curvature of the endovascular device.

In addition to providing lateral positional stability, filling the space in the aneurysm sac provides additional benefits. By slowing down flow, eliminating space or by adding a thrombogenic surface to the filler device(s), these devices can aid clot formation in the residual aneurysm sac, thereby decreasing the potential for endoleaks.

### BRIEF DESCRIPTION OF THE DRAWINGS

FIGS. 1A-1B illustrate the positioning of an endograft and the forces that act upon the graft over time. Movement of endografts downward accompanies lateral movement and buckling without considerable distal movement. Shown in FIG. 1A, the device has been implanted and the device has assumed a natural curvature. There is room for the device to move laterally due to lack of support. Shown in FIG. 1B, axial views of stent within aneurysm sac and lateral movement over time. Lack of lateral support allows the stent graft to move away from original position.

FIG. 2 shows aneurysm space filled with a stabilization system, thus preventing increase in curvature over what is present at initial deployment.

FIGS. 3A-3F illustrate specific stabilizing elements.

FIGS. 4A-4C illustrate a delivery system of the invention.

FIGS. 5A-5B are axial ultrasounds of aneurysms immediately and 6 months after endovascular repair. In FIG. 5A, the lateral position of the stent graft is stable and the patient had no evidence of downward displacement. In FIG. 5B the patient had clinical and radiographic migration while the limbs moved laterally as well.

FIG. 6A-6G show a deployment sequence using support balloons.

FIG. 7 shows the positioning of a thoracic aortic aneurysm, with stent and device of the invention.

FIG. 8A-8B illustrates optional attachment elements

FIG. 9 illustrates the movement of an endograft over time.

### DETAILED DESCRIPTION OF THE EMBODIMENTS

Downward displacement of stent-grafts can accompany lateral displacement of the endograft within the aneurysm sac, where lateral displacement is defined as a change in the three-dimensional position of the stent graft within the aneurysm sac. For the infrarenal aorta, stent graft migration (or the downward movement of the proximal portion of the stent graft) occurs when there is lateral movement of the stent graft within the aneurysm sac with little or no downward movement of the distal ends of the limbs.

The present invention provides methods and compositions for reducing the lateral displacement of an aortic endovascular device. A stabilization system comprising one or more stabilizing elements is inserted within the aneurysm space between an implanted device and the vessel wall. Filling this space prevents changes in curvature of the implanted endovascular device, and prevents longitudinal displacement, thereby providing for improved long-term stability and durability of endovascular repair. The invention includes any mechanism to maintain the lateral positional stability of any stent graft in any position within the vasculature. There are many different embodiments of the invention, some of which will be described below.

Before the present methods are described, it is to be understood that this invention is not limited to particular methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges, subject to any specifically excluded limit in the stated range.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a microsphere” includes a plurality of such microspheres and reference to “the stent” includes reference to one or more stents and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

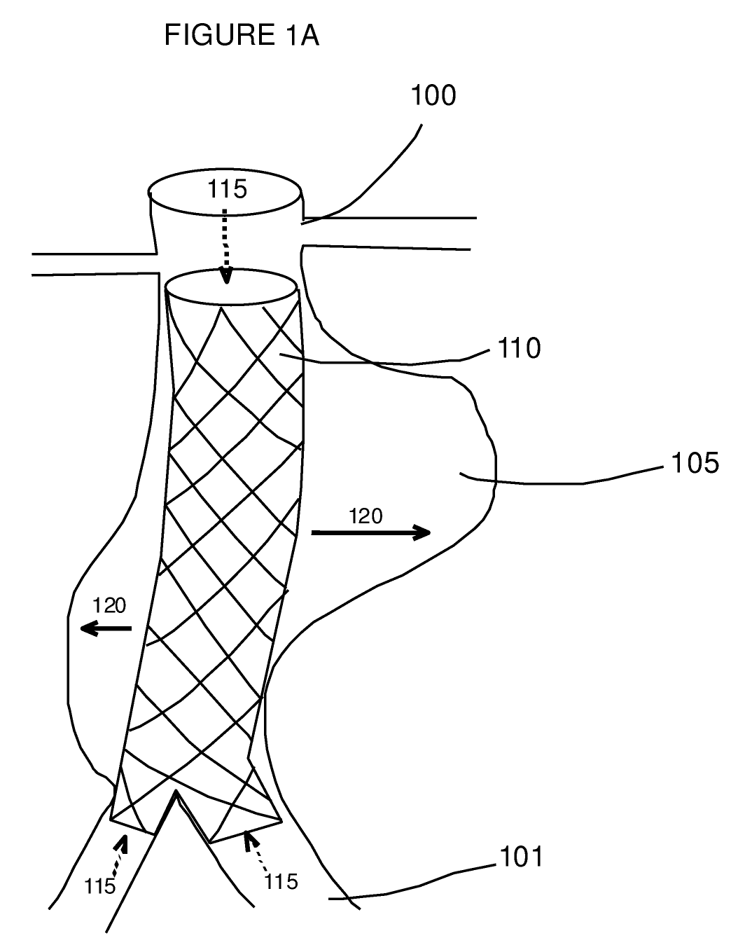

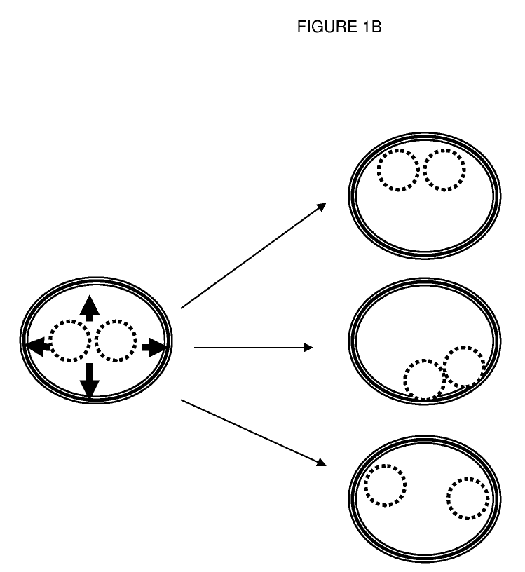

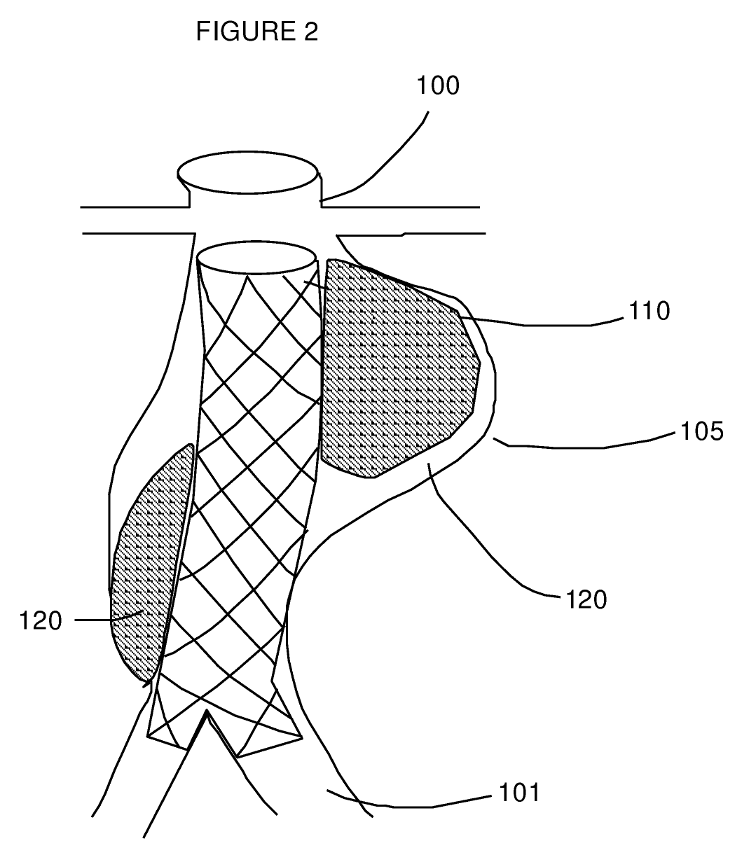

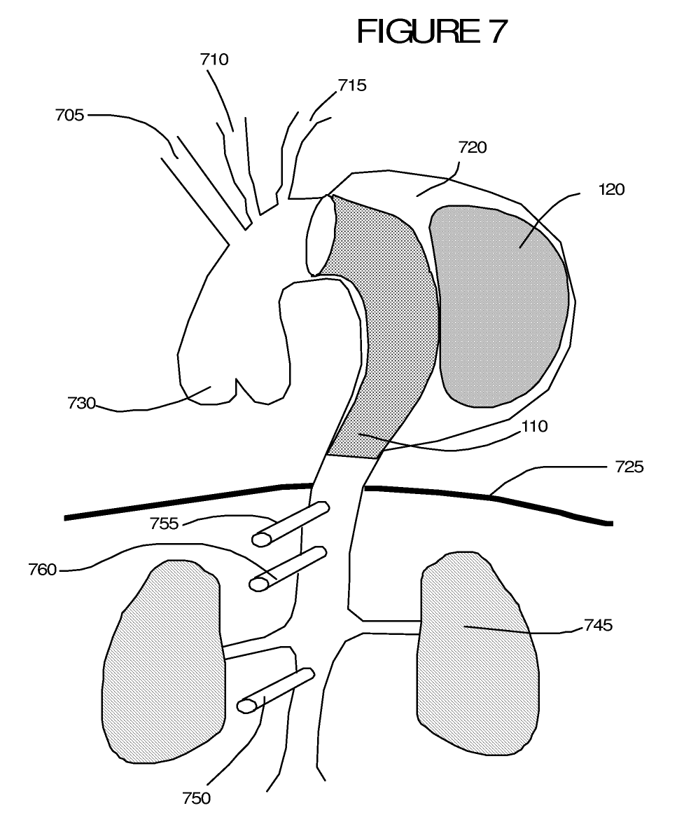

As shown in FIG. 1, an artery 100 with an aneurysm 105, for example between the iliac 101 and renal arteries, may be treated by insertion of a stent graft 110. The lateral movement 120 of the graft is associated with longitudinal movement 115, resulting in graft curvature and displacement. Shown in FIG. 1B are axial views of stent within aneurysm sac and lateral movement over time. Lack of lateral support allows the stent graft to move away from original position. As shown in FIG. 2, a stabilizing element 120 of the invention is used to full the aneurysm space, providing lateral stabilization. As shown in FIG. 7, the methods of the invention are also applied to the thoracic artery. The aneurysm 720, stent graft 110 and stabilizing element 120 depicted in the figure is in the thoracic aorta 730 between the braciocephalic artery 705, left common carotid artery 710, left subclavian artery 715; and the celiac trunk 755, superior mesenteric artery 760 and inferior mesenteric artery 750. The diaphragm 725 and kidneys 745 are also shown.

Stabilization System. As used herein, a stabilization system comprises one or more stabilization elements. When implanted, the stabilization system will fill sufficient space in the aneurysm to substantially prevent lateral movement of the endovascular device. Usually the stabilization system will contact the outer surface of the endovascular device, and the inner wall of the vessel at the maximum lateral dimension of the aneurysm. By filling the space between these surfaces, the stabilizing element supports the endovascular device and prevents it from movement and change in curvature. The elements of the stabilization system are usually not attached or anchored to the endovascular device or to the vessel wall.

The positioning of the stabilization system may be performed in conjunction with imaging, e.g. following the filling of the aneurysm space with a contrast agent. In this way, the stabilization system can be adjusted at the time of delivery to best fit the needs of the individual. Where a plurality of stabilizing elements are used, the number, size and position are desirably adjusted in accordance with the imaging information in order to appropriately fill the aneurysm space. Where a single stabilizing element is used, for example where the stabilizing element is a balloon, the degree to which the balloon is filled may be adjusted in accordance with imaging information. Where a plurality of elements are employed, the elements may be the same or different.

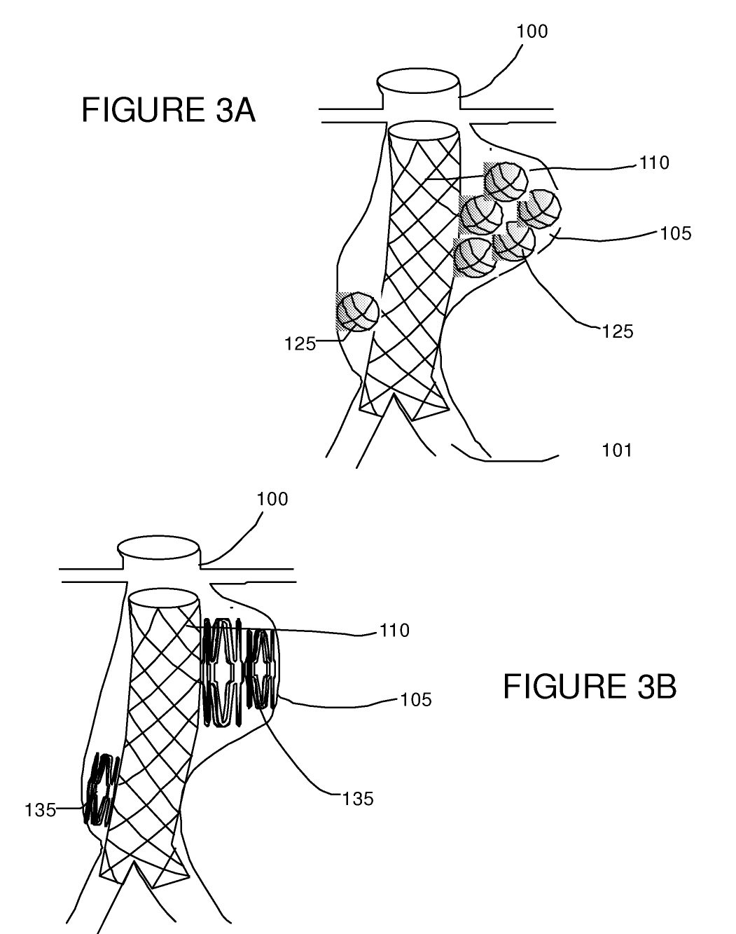

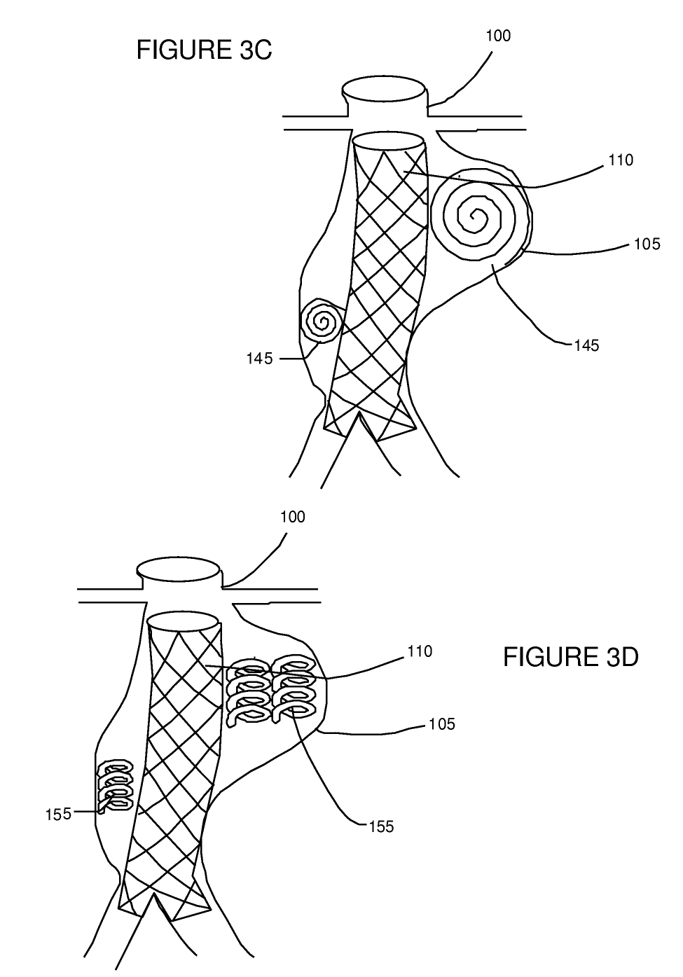

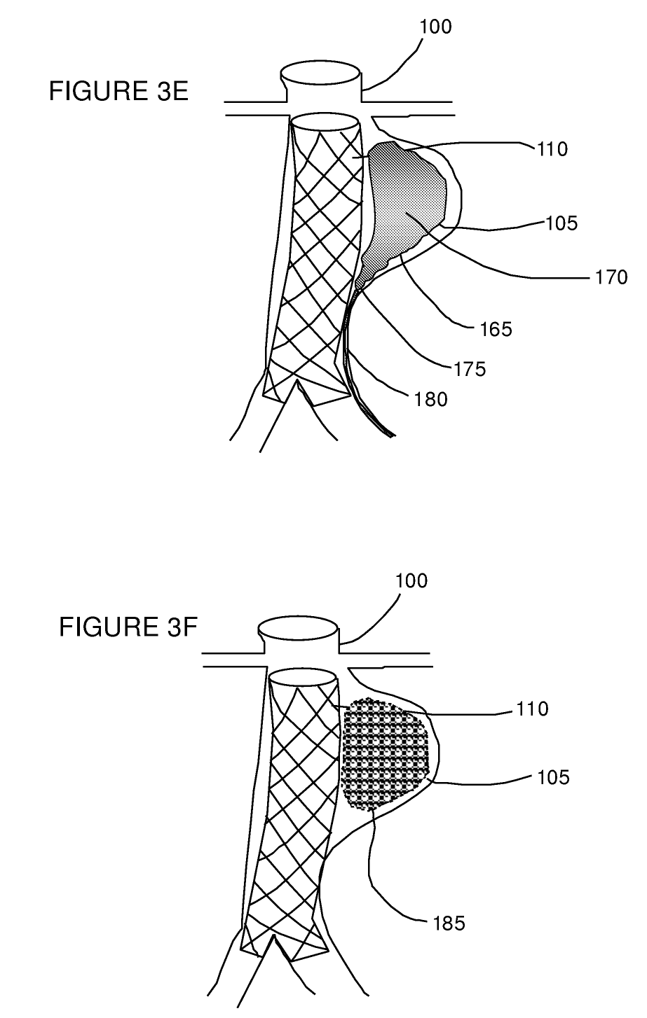

Stabilization Element. The methods of the invention encompass a variety of stabilizing elements, of which one or more are deployed at the aneurysm site. Exemplary elements are depicted in FIGS. 3A-3F. Expandable or self-expanding balloons, self expanding coils or stents, balloon expandable meshes, frames or coils, self expanding foams, fillers and adhesives contained in bags, envelopes or balloons of fabric or synthetic material are separate from the stent graft and are introduced into the aneurysm sac to fill the space within the sac which is not occupied by the stent graft. Depending on the needs of the individual, dimensions, type of structure and combinations thereof will be selected.

In one embodiment, as shown in FIGS. 3A-3D, the stabilizing element is an expandable, semi-rigid space-filling structure, which may be self-expandable, or balloon expandable. The structure will usually have dimensions of from about 0.25 cm diameter, about 0.5 cm diameter, about 1 cm diameter, about 2 cm diameter, and not more than about 7.5 cm diameter, usually not more than about 5 cm diameter. It will be understood by those of skill in the art that various geometric shapes are known and are commercially available, including rods, spheroid shapes, toroids, cylinders, coils, springs, and the like, may be used. Depending on the spatial requirements of the individual aneurysm sac, one or more structures may be deployed, which may be 2, 3, 4, 5, 6, and not more than about 12. The structures may be deployed at one or more sites in the aneurysm sac, depending on the requirements of the individual. The structure is optionally filled with a biocompatible matrix.

As shown in FIG. 3A, an aneurysm sac 105 bounded by the vessel wall 100 is lateral to a stent graft 110. The space within the sac is shown to be filled with semi-rigid frames 125. The frames may be a toroid, spheroid, cylindrical, etc. shape. In some embodiments, the frames are an occlusion device, for example an AMPLATZER™ device (AGA Medical Corporation, for example as described in U.S. Pat. No. 7,001,409, “Intravascular flow restrictor”; U.S. Pat. No. 6,682,546, “Intravascular occlusion devices”; U.S. Pat. No. 6,638,257, “Intravascular flow restrictor”; U.S. Pat. No. 6,599,308, “Intravascular occlusion devices”; U.S. Pat. No. 6,506,204, “Method and apparatus for occluding aneurysms”, herein incorporated by reference), in which the frame is a metal fabric formed of a plurality of resilient strands is provided, with the wires being formed of a resilient material which can be heat treated to substantially set a desired shape. This fabric is then deformed to generally conform to a molding surface of a molding element and the fabric is heat treated in contact with the surface of the molding element at an elevated temperature. The time and temperature of the heat treatment is selected to substantially set the fabric in its deformed state. After the heat treatment, the fabric is removed from contact with the molding element and will substantially retain its shape in the deformed state. The fabric so treated defines an expanded state of a medical device which can be deployed through a catheter into a channel in a patient's body. Such devices are formed of a metal fabric and have an expanded configuration and a collapsed configuration. The devices are collapsed for deployment through a catheter and, upon exiting the distal end of the catheter in a patient's channel, will resiliently substantially return to their expanded configuration. For example, the device may form a tubular braid, or may take the form of a flat woven sheet, knitted sheet or the like.

Where the device is a self-expanding structure, the metal is selected to be both resilient and can be heat treated to substantially set a desired shape. Materials which are believed to be suitable for this purpose include a cobalt-based low thermal expansion alloy referred to in the field as Elgiloy, nickel-based high-temperature high-strength “superalloys” commercially available from Haynes International under the trade name Hastelloy, nickel-based heat treatable alloys sold under the name Incoloy by International Nickel, and a number of different grades of stainless steel. The important factor in choosing a suitable material for the wires is that the wires retain a suitable amount of the deformation induced by a molding surface when subjected to a predetermined heat treatment. One class of materials which meet these qualifications are so-called shape memory alloys. Such alloys tend to have a temperature induced phase change which will cause the material to have a preferred configuration which can be fixed by heating the material above a certain transition temperature to induce a change in the phase of the material. When the alloy is cooled back down, the alloy will “remember” the shape it was in during the heat treatment and will tend to assume that configuration unless constrained from so doing. One particularly preferred shape memory alloy for use in the present method is nitinol, an approximately stoichiometric alloy of nickel and titanium, which may also include other minor amounts of other metals to achieve desired properties. NiTi alloys such as nitinol, including appropriate compositions and handling requirements, are well known in the art and such alloys need not be discussed in detail here.

The filaments or metal wires of the frame may have any cross-sectional geometry, e.g. square, round oval, triangular, etc. The springs and arms may be made of the same or different material, and combinations of materials may be used. Diameter of the filament or formed pieces will vary widely depending on the structure of the frame. A filament may range from about 0.05 mm to 0.15 mm, to as large as about 0.5 mm diameter or more. The cross-section of a filament need not be constant along its entire length, but may include portions having a larger or smaller cross-section as desired.

Where the structure is balloon expandable, the material need not be formed of a shape memory metal, but may be formed of a variety of biocompatible materials, including, for example, stainless steel, titanium, tantalum, gold, platinum, copper and the like, as well as alloys of these metals. Low shape memory plastic may also be used. Examples of shape memory plastics may be found in U.S. Pat. No. 5,506,300; U.S. Pat. No. 5,665,822, and biodegradable shape memory plastics, e.g. U.S. Pat No. 6,160,084.

Stents, which may be self-expanding or balloon expandable are also suitable for use as stabilizing elements. As used herein, the term stent is used as is known in the art, to refer to a prosthesis which can be inserted and held, when desired, in a lumen of a vessel or organ in the body. As shown in FIG. 3B, one or more stents of conventional design 135 are implanted outside of the primary stent, where the space-filling stent 135 is in a positional lateral to the primary stent, i.e. alongside and extending into the aneurysm sac. The stent may be of any design or material, as known and described in the art. Materials commonly used in stent construction include biologically compatible metals, e.g. stainless steel, titanium, tantalum, gold, platinum, copper and the like, as well as alloys of these metals; low shape memory plastic; a shape-memory plastic or alloy, such as nitinol; and the like. Also useful are polymeric biodegradable stents, anastomotic devices, and scaffolds, including synthetic biodegradable or bioerodible porous scaffolds.

The dimensions of the stent for use in the methods of the invention will be selected based on the individual needs of the patient, and in an individual multiple sizes may selected. The stent will usually be at least about 1.5 cm in length, usually at least about 2.5 cm in length, and not more than about 10 cm in length. The stent will have a diameter prior to expanding of at least about 0.1 cm, usually around about 0.5 cm; and after expansion has a diameter of at least about 1 cm, usually at least about 2.5 cm, and not more than about 5 cm. The stent is optionally crimped onto a balloon, which when inflated will expand the stent to its full diameter.

As an alternative to a stent, the stabilizing element may be a coil 145, as shown in FIG. 3C; or a coil 155, as shown in FIG. 3D. The materials for these elements will be the same as those for stents and frame structures.

Any one of the above described structures may be coated with a medium. For example the structure may be covered with a polymer such as ethylene vinyl alcohol copolymer (EVOH); polybutylmethacrylate; poly(hydroxyvalerate); poly(L-lactic acid); polycaprolactone; poly(lactide-co-caprolactone); poly(lactide-co-glycolide); poly(hydroxybutyrate); poly(hydroxybutyrate-co-valerate); polydioxanone; polyorthoester; polyanhydride; poly(glycolic acid); poly(D,L-lactic acid); poly(glycolic acid-co-trimethylene carbonate); polyphosphoester; polyphosphoester urethane; poly(amino acids); cyanoacrylates; poly(trimethylene carbonate); poly(iminocarbonate); copoly(ether-esters); polyalkylene oxalates; polyphosphazenes; biomolecules; polyurethanes; silicones; polyesters; polyolefins; polyisobutylene and ethylene-alphaolefin copolymers; acrylic polymers and copolymers; vinyl halide polymers and copolymers; polyvinyl ethers; polyvinylidene halides; polyacrylonitrile; polyvinyl ketones; polyvinyl aromatics; polyvinyl esters; copolymers of vinyl monomers with each other and olefins, acrylonitrile-styrene copolymers, ABS resins, and ethylene-vinyl acetate copolymers; polyamides; alkyd resins; polycarbonates; polyoxymethylenes; polyimides; polyethers; epoxy resins; polyurethanes; rayon; rayon-triacetate; cellulose; cellulose acetate; cellulose butyrate; cellulose acetate butyrate; cellophane; cellulose nitrate; cellulose propionate; cellulose ethers; amorphous Teflon; and carboxymethyl cellulose or a combination thereof, but not limited to these polymers.

In a further embodiment of the invention the structure is a drug eluting; or drug coated. In contrast to conventional drug-eluting devices, for the purposes of the present invention it is desirable to occlude the space in which the device is situated. Drugs of interest include agents that increase fibrosis, or proliferative agents that increase the growth of endothelial or smooth muscle cells. Examples of biologically active agents include, but are not limited to, drugs, antibodies, growth factors, chimeric proteins or decoy inhibitors, genetic material delivered by agents such as viral gene transfer vectors or non-viral transfection methods, cells oligonucleotides or small interfering RNA molecules. The foregoing substances are listed by way of example and are not meant to be limiting. Other therapeutic substances which are currently available or that may be developed in the future are equally applicable.

In an alternative embodiment, as shown in FIGS. 3E-3F, the stabilizing element is an expandable, conformable space-filling structure. In one such embodiment, as shown in FIG. 3E, the stabilizing element comprises one or more balloons 165, comprising a port or valve 175 which is attachable to a filling tube or conduit 180, and filled with a suitable medium 170. In other embodiments, for example as shown in FIG. 3F, the stabilizing element is a sponge 185, or other conformable, porous element. A sponge may comprise coatings or other materials that increase fibrosis.

As used herein, a balloon refers to a cylindrical element defining a hollow space, which can be delivered in an unexpanded state, and will expand when filled with medium, e.g. saline, gel, etc. While many balloons are formed of a flexible material, e.g. silicone, etc., such is not required, and a balloon may alternatively be formed of a woven permeable or impermeable fabric, such polyester, polytetrafluoroethylene (PTFE), silicones, and urethanes. Where the balloon is a more permeable fabric, it will generally be filled with gel or settable fluid, while impermeable materials may be filled with medium that remains fluid. Various portions of the balloon assembly may be radiolucent in order to make it easier to locate during the endoscopic procedures. Alternatively, the balloon assembly may have radiolucent markers added thereto for purposes of locating and maneuvering the assembly during the process steps of the invention. Further, the balloon structure may utilize silicone pigments or expansion fluid which is radiolucent.

Various biologically compatible media are useful for filling the balloon at the site of the aneurysm sac, where the balloon will be filled with a volume sufficient to substantially fill the aneurysm sac space, and prevent lateral movement of the primary stent.

Biocompatible, non-biodegradable polymers of interest for filling the balloon include polycarbamates or polyureas, particularly polyurethanes, polymers which may be cross-linked to produce non-biodegradable polymers such as cross-linked poly(vinyl acetate) and the like. Also of particular interest are ethylene-vinyl ester copolymers having an ester content of 4 to 80% such as ethylene-vinyl acetate (EVA) copolymer, ethylene-vinyl hexanoate copolymer, ethylene-vinyl propionate copolymer, ethylene-vinyl butyrate copolymer, ethylene-vinyl pentantoate copolymer, ethylene-vinyl trimethyl acetate copolymer, ethylene-vinyl diethyl acetate copolymer, ethylene-vinyl 3-methyl butanoate copolymer, ethylene-vinyl 3-3-dimethyl butanoate copolymer, and ethylene-vinyl benzoate copolymer. Additional naturally occurring or synthetic non-biodegradable polymeric materials include poly(methylmethacrylate), poly(butylmethacrylate), plasticized poly(vinylchloride), plasticized poly(amides), plasticized nylon, plasticized soft nylon, plasticized poly(ethylene terephthalate), natural rubber, silicone, poly(isoprene), poly(isobutylene), poly(butadiene), poly(ethylene), poly(tetrafluoroethylene), poly(vinylidene chloride), poly(acrylonitrile, cross-linked poly(vinylpyrrolidone), poly(trifluorochloroethylene), chlorinated poly(ethylene), poly(4,4′-isopropylidene diphenylene carbonate), vinylidene chloride-acrylonitrile copolymer, vinyl chloride-diethyl fumarate copolymer, silicone, silicone rubbers (especially the medical grade), poly(dimethylsiloxanes), ethylene-propylene rubber, silicone-carbonate copolymers, vinylidene chloride-vinyl chloride copolymer, vinyl chloride-acrylonitrile copolymer, vinylidene chloride-acrylonitrile copolymer, poly(olefins), poly(vinyl-olefins), poly(styrene), poly(halo-olefins), poly(vinyls), poly(acrylate), poly(methacrylate), poly(oxides), poly(esters), poly(amides), and poly(carbonates). Two component polymers approved for use in humans are preferred.

Temperature setting polymers are also of interest, where, for example, a polymer may be delivered in a temperature regulated catheter where the polymer is flowable at the catheter temperature, but polymerizes at body temperature. Alternatively, a heating or cooling control may alter the temperature of the catheter at the terminus, in order to set the polymer as it is delivered to the site.

Balloons will comprise a valve, or port, which may be a self-sealing port, for communication with a tube or conduit extending from outside the body of the patient. The conduit is arranged to carry a fluid, e.g., a gel or saline solution, which is carried therein for introduction through the port or valve into the interior of the balloon to inflate the balloon. Preferably the balloons interior is filled sufficiently to cause the outer wall of the balloon to extend outward to substantially fill the aneurysmal space laterally. Once the balloon has been expanded sufficiently to fill the aneurysmal space the conduit can be removed, whereupon the self-sealing port or valve closes to trap the fluid within the balloon, thereby keeping the balloon inflated. As an alternative to a self-sealing port, the balloon may have a valve, for example a ball valve, which is preferably constructed of a rigid material, for example titanium, a polymeric material or like material.

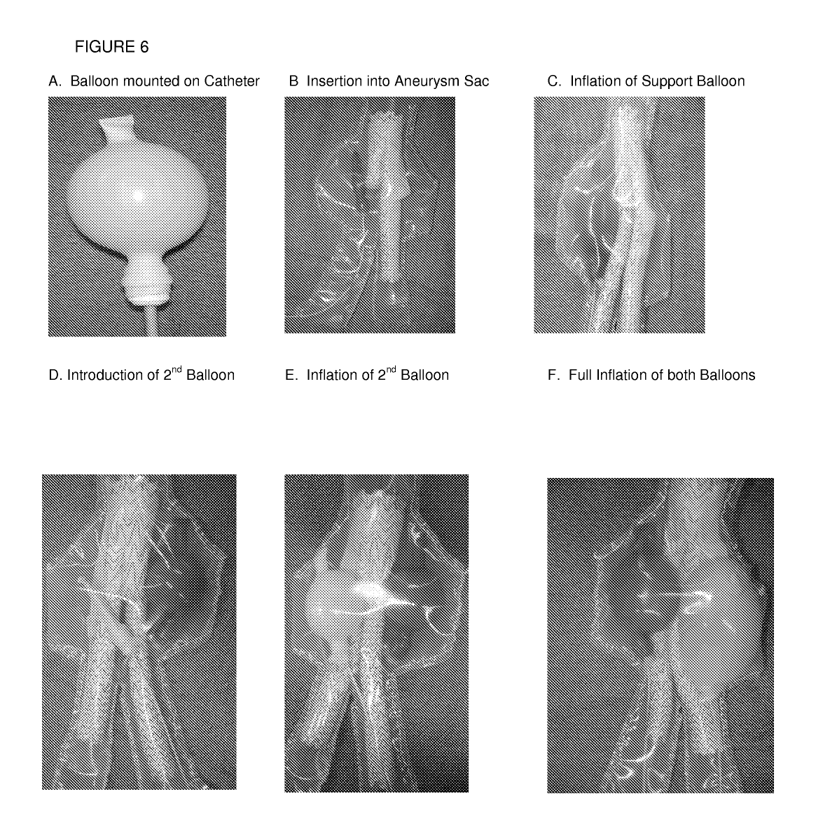

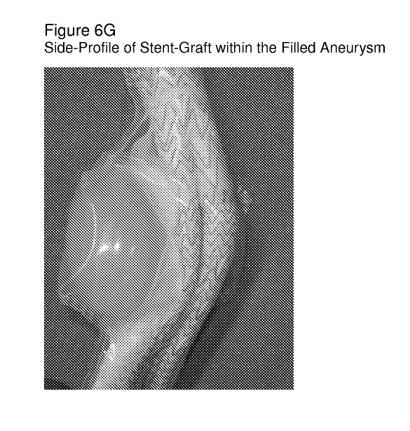

If the balloon becomes incompetent or erodes after implantation, the injectate has already hardened and does not provide a risk of dislodgement or embolization. This mechanism has been tested on an infrarenal aortic aneurysm model. The photos are shown in FIGS. 6A-6G.

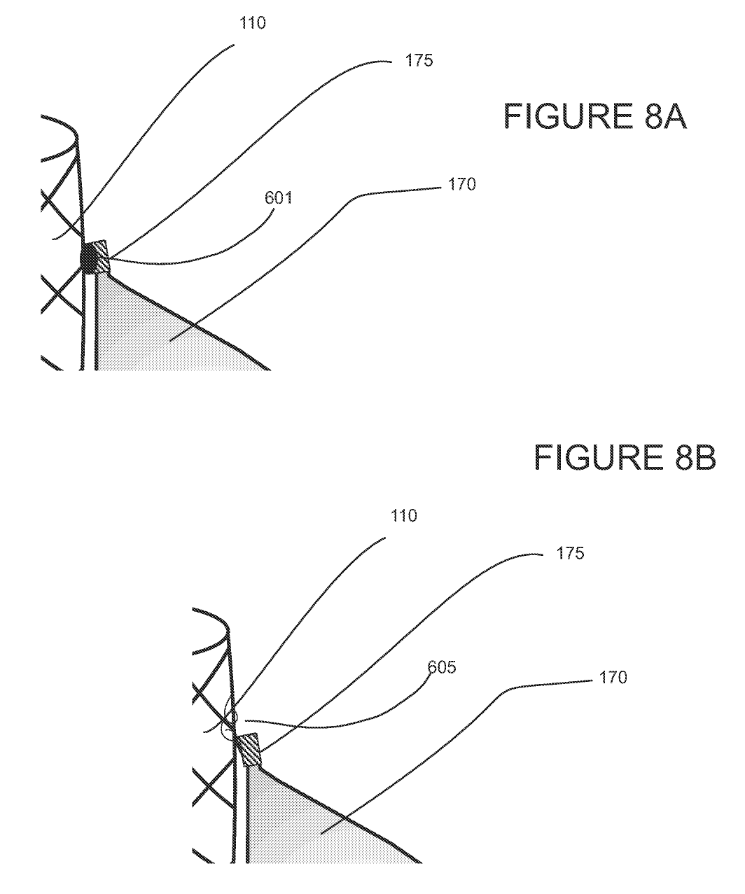

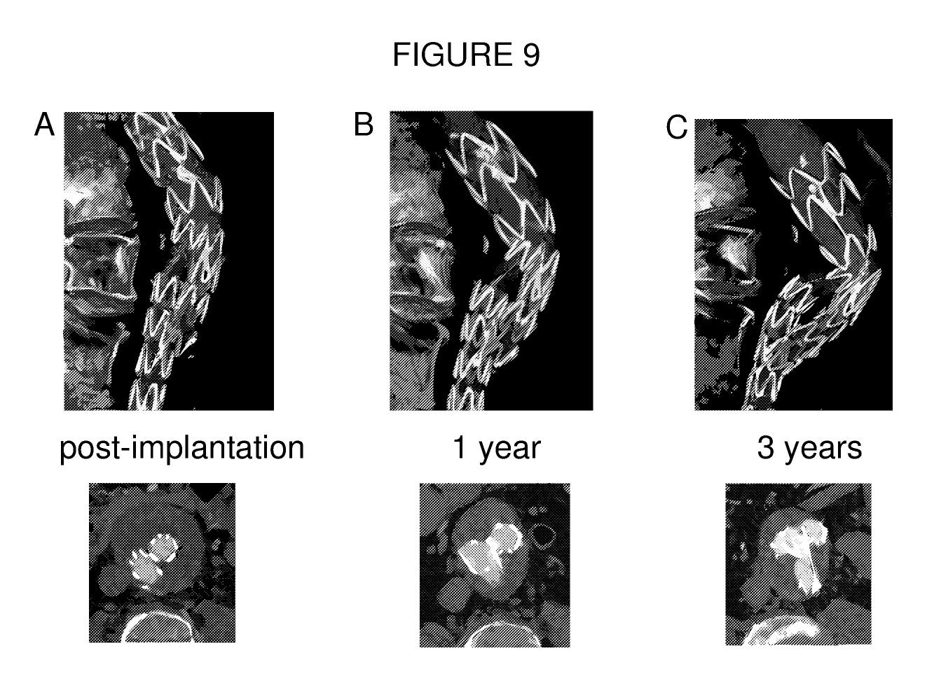

In some embodiments the stabilizing element is attached to the endograft, which attachment may be performed after deployment of the endograft. The stabilizing element is attached to the endograft by any convenient method, e.g. with glue, barbs, hooks, etc. FIG. 8 illustrates options for an attachment element. For convenience the attachment element is illustrated with a balloon stabilizing element, however it will be understood by those of skill in the art that the attachment element design may be used with any of the previously described stabilizing elements.

Where a glue is used, the catheter may be modified to provide delivery of the glue. For example, a catheter may separately deliver each component of a two component glue, which is then mixed in situ. Where the stabilizing element is attached by an attachment element, for example a barb, hook, Velcro, etc., the attachment element may make use of existing sites on the endograft to create an attachment point. As shown in FIG. 8A, an attachment element may be a glue spot 601 on a convenient point on the stabilizing element, e.g. the valve of a balloon, a wire of a stent, and the like. Alternatively, as shown in FIG. 8B, an attachment element may be a wire 605 that hooks or threads through the endograft, and that is threaded or otherwise attached to the stabilizing element.

Endovascular device. The methods of the invention may be used with any endovascular device, particular devices for use in aortic and thoracic aneurysms. Such devices typically comprise a stent and a graft. The stent graft may be bifurcated, for example comprising an aortic stent and one or two iliac stents. Such bifurcated stents are known in the art and available in a variety of configurations, e.g. in a modular design, a unitary design, etc.

Stents can be self-expanding or expandable via an internal expanding device such as a balloon. For example, stents may be self-expandable and comprised of a shape memory alloy, where the alloy can be deformed from an original, heat-stable configuration to a second, heat-unstable configuration. The application of a desired temperature causes the alloy to revert to an original heat-stable configuration. A stent may be fabricated from a single piece of alloy tubing, where the tubing is laser cut, shape-set by placing the tubing on a mandrel, heat-set to its desired expanded shape and size and electropolished. The stent will be sized to fit the vessel for which it is intended. It typically is desirable to “oversize” the stent to assure a good seal and engagement within the aorta, where a minimum of about 2 mm oversize is preferred. The stent is optionally provided with barbs, hooks and the like to increase the mechanical fixation of the stent to the aorta, although such is not necessary.

The graft components have a generally tubular shape, and are made of materials which include woven and knitted materials comprising polyester, polytetrafluoroethylene (PTFE), silicones, and urethanes. The materials may be porous or nonporous and may be opaque to X-rays. Preferred materials include polyester fabric, for example DACRON™, TEFLON™, or other suitable fabric. The graft material is attached to the stent via sutures or other suitable attachment means. Attachment is typically done before the system is loaded into a delivery device.

Delivery Device. Typically, an endoluminal device, such as a stent-graft deployed in a blood vessel at the site of a stenosis or aneurysm, is implanted endoluminally by minimally invasive techniques in which the device, restrained in a radially compressed configuration by a sheath or catheter, is delivered by a delivery system to the site where it is required. Typically a guide wire is first introduced into the vasculature, and threaded through the vasculature to the site for deployment. One or more catheters may then be guided to the site along the wire.

In the methods of the present invention it is often desirable to image the aneurysm site by delivery of contrast medium for X-ray, CT imaging, etc., e.g. through a catheter which may be a catheter for device delivery. In order to be able to regulate the position of the catheter in the blood vessel a contrast ring may be provided, this ring being made of a material which, under X-ray radiation, provides a contrast with the environment so that the location of the catheter in its introduced state is detectable. Suitable X-ray contrast producing materials are precious metals, gold being particularly preferred. The catheter system may also include an imaging system having optical fibers or the like extending along the length of the tube and terminating at a viewing window in the insertion tube's distal end. The imaging system conveys an image from a viewing window to an eyepiece on the headpiece, or to a monitor, so that the user can see into a selected body cavity during an endoscopic procedure. Through manipulation of the controls, an operator can cause the distal end of the insertion tube to become substantially linear, or to take a curved shape to selectively position the viewing window.

The delivery system may enter the body from an access location outside the body, such as through the patient's skin, or by a cut down technique in which the entry blood vessel is exposed by minor surgical means. The term “proximal” as used herein refers to portions of the stent or delivery system relatively closer to this access location, whereas the term “distal” is used to refer to portions farther from the access location.

When the delivery device has been threaded into the body lumen to the stent deployment location, the introducer is manipulated to cause the stent to be ejected from the surrounding sheath or catheter in which it is restrained, or alternatively the surrounding sheath or catheter is retracted from the stent, whereupon the stent expands to a predetermined diameter at the deployment location, and the introducer is withdrawn. Stent expansion may be effected by spring elasticity, balloon expansion, or by the self-expansion of a thermally or stress-induced return of a memory material to a pre-conditioned expanded configuration.

Various delivery devices find use with the methods of the present invention, which will device will vary depending on whether the stabilizing device is delivered independently of the stent graft; and whether the stabilizing device requires a filling tube or conduit for balloon inflation.

The catheter may be made of materials conventional and suitable in the manufacture of catheters. Such materials are neutral with regard to body fluid, they may be sterilized without problems and they are sufficiently elastic but on the other hand also sufficiently rigid to be introduced in blood vessels. Suitable materials are polyolefins, polyfluorated carbohydrate polymers, synthetic rubbers, polyvinyl fluorides and the like. Particularly preferred materials for catheters are silicon rubber and implantable polyvinyl chloride. For the hose connection basically comparable materials are suitable, especially however polyolefins, fluorated carbohydrate polymers or polyvinyl chloride for the hoses. Where applicable, hose pumps, including peristaltic pumps are commercially available.

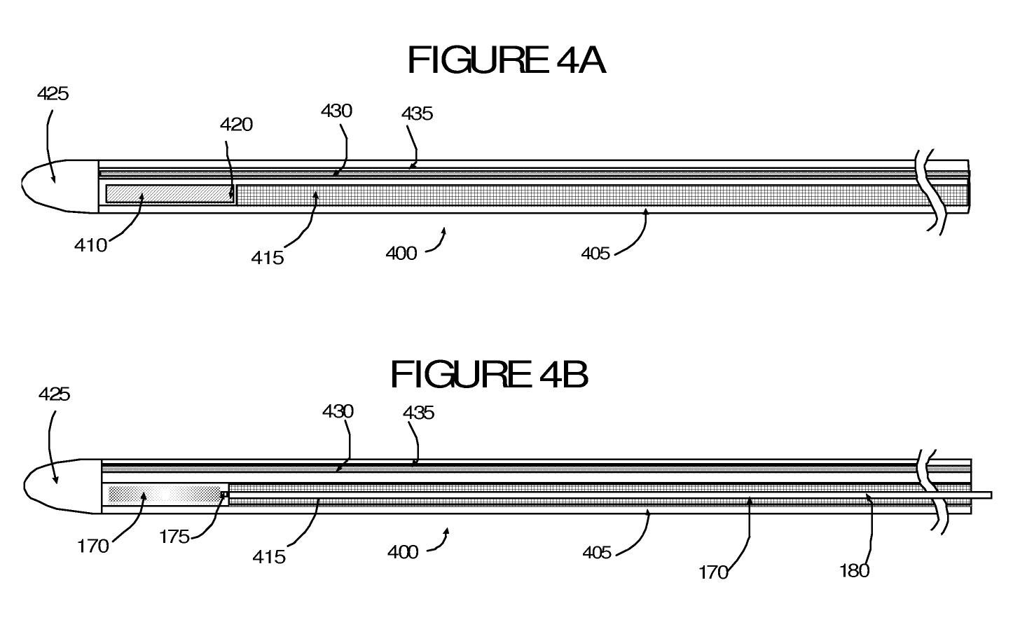

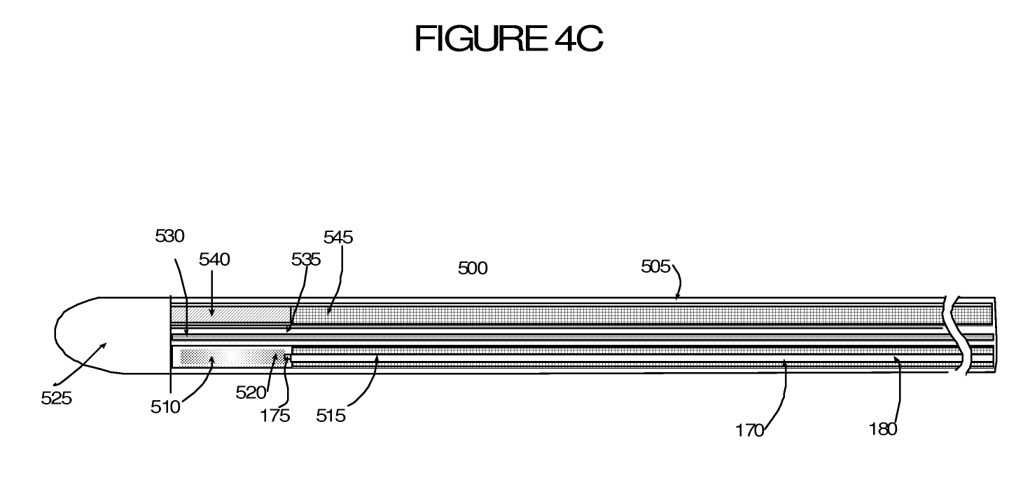

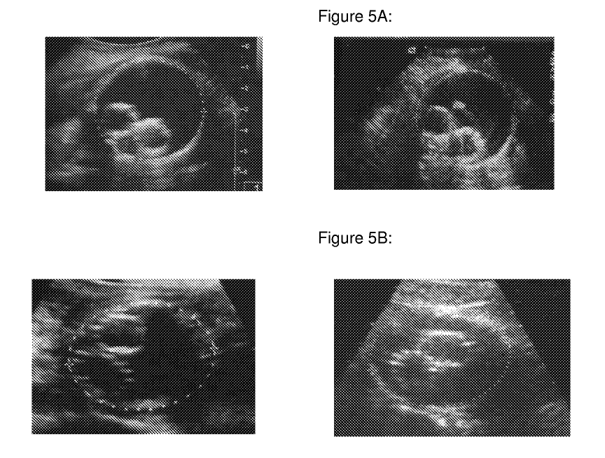

The stabilizing element(s) can be introduced before, during or after deployment of the stent-graft. Where the stabilizing device is delivered independently of the stent graft, for example to an existing stent graft, or in an independent procedure immediately following deployment of a stent graft, a single lumen catheter, for example as shown in FIG. 4A may be utilized. In such embodiments, a guide wire may be introduced specifically for delivery of the stabilization element, or may be left in place after deployment of the stent graft. FIG. 4A shows a preloaded delivery system 400 comprising an outer sheath 405, at least one compressed stabilizing element of the invention 410 loaded therein, and a stabilizer 415 loaded adjacent to the proximal end 420 of the stabilizing element. The delivery system may also comprise a catheter tip 425 at its distal end attached to an internal shaft 430 that runs through the delivery system through inner lumen 435. The internal shaft 430 may guide the delivery system through the body lumen over a guide wire (not shown) to the area to be stabilized.

In some embodiments the internal shaft 430 is adapted to deliver medium for balloon expansion. Balloon expansion may be utilized in expanding a compressed device, e.g. stent or other non-self-expanding device. Alternatively, where the stabilizing element itself is a balloon, the balloon expansion is used to fill the stabilizing element. In such embodiments, as shown in FIG. 4B, the delivery device is loaded with a balloon 165 comprising a port or valve 175 attached to a filling tube or conduit 180, which is filled with a suitable medium 170 that is pumped into the balloon. The filling tube may run through the stabilizer 415.

A standard deployment technique comprises maneuvering the introducer to a desired deployment location and retracting outer sheath 405 so that the stabilizing element is deployed beginning at its distal end and ending at its proximal end. The stabilizer 415 stabilizes or prevents retraction of the stabilizing element when sheath 405 is retracted, thus effecting deployment of the device into a desired location by forcing relative movement between the sheath and the device. Where a balloon deployment is involved, the medium 170 is pumped or otherwise pressurized to fill the balloon, which is released when appropriately full. If desired, air, saline or other retrievable material can be injected into deflatable balloon(s) or spaces to confirm location and secure positioning prior to filling with biocompatible material capable of hardening. The connection of the catheter to the hose pump is by way of hose connections which are connected to a member at the rear end of the catheter.

In other embodiments of the invention, the stabilizing element is delivered as the same time as the endoluminal device. In such embodiments, it may be desirable to utilize a double lumen catheter in order to minimize flow disturbances within the blood vessel. In some such embodiments, the stabilization element is delivered in a catheter with a stent graft, where the stent graft may be a primary aortic device, or where the stent graft is a modular device, the stent may be a bifurcation module, e.g. an iliac module. Where the delivery device comprises a primary aortic device and a stabilization element, the aortic device will usually be deployed first, followed by deployment of the stabilization element. Where the delivery device comprises an iliac module, the stabilization element will usually be deployed following the aortic module, and before deployment of the iliac module.

For example, in a modular bifurcated aortic stent graft, after deployment of the main aortic body of a modular stent graft device, an open limb of an iliac artery is canulated with a guide wire. Before introduction of the iliac module of the stent graft, the iliac access catheter is used to introduce the stabilization element into the aneurysm sac around the primary aortic module, in order to maintain the lateral position of the stent graft within the aneurysm sac.

Alternatively, the contralateral iliac limb is partially or fully deployed leaving a guide wire between the deployed iliac limb and the iliac wall. This guide wire leads into the aneurysm sac, outside the deployed stent graft. A similar guide wire can be left in place, leading into the aneurysm sac, outside the stent graft on the primary module side. The guide wire (s) leading into the aneurysm sac can be used to introduce delivery catheters into the aneurysm sac, through which the stabilization elements are deployed.

A delivery device suitable for coordinate deliver of a stent graft and a stabilization element is shown in FIG. 4C. The figure specifically illustrates a catheter loaded with a balloon stabilization element, as shown in FIG. 4B, but it will be understood by one of skill in the art that other stabilization elements are readily delivered by a double lumen catheter as well, and that the catheter will be modified appropriately.

As shown in FIG. 4C, the preloaded delivery system 500 comprises an outer sheath 505, at least one compressed stabilizing element of the invention 510 loaded therein, and a stabilizer 515 loaded adjacent to the proximal end 520 of the stabilizing element. The balloon stabilizing element comprises a port or valve 175 attached to a filling tube or conduit 180, which is filled with a suitable medium 170 that is pumped into the balloon. The delivery system may also comprise a catheter tip 525 at its distal end attached to an internal shaft 530 that runs through the delivery system through inner lumen 535. The internal shaft 530 may guide the delivery system through the body lumen over a guide wire (not shown) to the area to be stabilized. In the second lumen of the catheter is a preloaded, compressed endovascular device 530 and a stabilizer 545 loaded adjacent to the proximal end of the device.

Insertion of the stabilization system may occur via the delivery system shown in FIGS. 4A-4C. The artery is usually entered by an arterial incision where the vessel is close to the undersurface of the skin, e.g. in the thigh. A guide wire is first endoluminally placed, using conventional techniques to a position in the patient's aorta, above or below an aortic aneurysm such as depicted in FIG. 1. The delivery system of this invention is guided into the aneurysm along this guide wire. The guide wire remains in a fixed position throughout the endoluminal procedure. Conventional angiography techniques are employed to identify the aneurysm and the position of key anatomical structures such as the renal arteries.

### Kits

The methods of this invention are preferably conducted by using kits of parts comprising two or more of the components necessary for endovascular device stabilization. For example, in one embodiment, this kit comprises the following components: (a) a stabilization system comprising one or more stabilization elements; and (b) a catheter suitable for delivering the stabilization system. The kit may further comprise an imaging system for imaging the aneurysm space in conjunction with deployment of the stabilization system. Where the stabilization system comprises one or more balloons, the kit may comprise a biocompatible fluid suitable for filling the balloon, and a catheter suitable for delivering the fluid composition to the aneurysm site. The kit may further comprise a catheter suitable for delivering an endovascular prosthesis to the aneurysm. In some embodiments of the invention, a double lumen catheter suitable for delivering both the endovascular prosthesis and the stabilization system is provided.

### Utility

The methods described herein are useful in reducing or eliminating lateral displacement in a stent graft utilized for repair of an aneurysm, thereby reducing or eliminating the resulting device migration, type I and III endoleaks, stent fractures, fabric tears, and modular disconnections. Accordingly, these methods find use in human and other mammalian subjects requiring repair of such aneurysms. It is contemplated that the procedures set forth above can be employed for stabilization of an endovascular prosthesis at vascular sites other than the abdominal aorta. Such prostheses could be used to repair aneurysms and other vascular diseases at vascular sites.

Long-term clinical assessment of patients treated with endografts include periodic imaging of the aneurysm and endograft using CT or ultrasound. While most studies focus on longitudinal positional stability of the proximal portion of the endograft, we have evaluated the lateral positional stability of the endograft within the aneurysm sac. These studies show that lateral movement of the midportion of the endograft at one year corresponds to a high rate of late adverse clinical events such as Type I endoleak, risk of rupture and need for secondary procedures and surgical conversion. Lateral endograft movement within the aneurysm sac is at least as good a predictor of late adverse events as longitudinal migration. Thus, stabilization of the mid-portion of the endograft to prevent lateral movement will help prevent endograft displacement and late adverse events. Curved geometries accentuate the lateral displacement forces acting on endografts and large diameter aneurysms are particularly vulnerable to sideways movement of the endograft. Endografts are unstable over time in such geometries and a mechanism to stabilize the midportion of the endograft in the large aneurysm sac will significantly improve long term results. This invention provides the means to prevent movement of the endograft in the aneurysm sac.

What is claimed is:

1. A method for lateral stabilization of an endovascular device within an aneurysm space, the method comprising:
inserting one or more stabilization elements within the aneurysm space between an implanted device and the vessel wall, wherein the stabilization elements prevent longitudinal displacement of the endovascular device.

2. The method of claim 1, wherein the one or more stabilization elements are delivered under imaging guidance using a directional deflectable catheter system.

3. The method of claim 2, wherein the one or more stabilization elements are delivered over a wire from above the endovascular device.

4. The method of claim 2, wherein the one or more stabilization elements are delivered parallel to the endovascular device after full deployment of the device.

5. The method of claim 2, wherein the endovascular device is a modular bifurcated aortic stent graft.

6. The method of claim 5, wherein after deployment of a main aortic body of the modular stent graft device, the method comprises the steps of:
canulating an open limb of an iliac artery with a guide wire;
introducing, with an iliac access catheter guided by the guide wire, one or more stabilization elements within the aneurysm space between the main aortic body of the modular stent graft device and the vessel wall;
introducing the iliac module of the stent graft with an iliac access catheter.

7. The method of claim 5, wherein after deployment of a main aortic body of the modular stent graft device, the method comprises the steps of:
canulating an open limb of an iliac artery with a guide wire;
introducing the iliac module of the stent graft with an iliac access catheter while leaving a guide wire between the deployed iliac limb and the iliac wall,
introducing, with an iliac access catheter guided by the guide wire, one or more stabilization elements within the aneurysm space between the main aortic body of the modular stent graft device and the vessel wall.

8. The method of claim 2, wherein the one or more stabilization elements are delivered by a single lumen catheter.

9. The method of claim 6 or claim 7, wherein the one or more stabilization elements are delivered by a double lumen catheter comprising the iliac module of the stent graft.

10. The method of claim 1, wherein the stabilizing element is not anchored or fixed to the endovascular device.

11. The method of claim 1, wherein the stabilizing element is fixed to the endovascular device.

12. The method of claim 10, wherein the stabilization element is an expandable, semi-rigid space-filling structure.

13. The method of claim 12, wherein the stabilization element is of a rod, spheroid, toroid, cylindrical, coil, or spring geometry.

14. The method of claim 13, wherein the stabilization element is formed of a self-expanding material of from about 0.25 cm to about 5 cm diameter.

15. The method of claim 13, wherein from 1 to 12 elements are inserted in the aneurysmal space.

16. The method of claim 12, wherein the structure is formed of a shape memory metal.

17. The method of claim 12, wherein the space filling structure is filled with a biocompatible matrix or cells.

18. The method of claim 10, wherein the one or more stabilization elements are balloons.

19. The method of claim 18, wherein the balloons are filled with a settable biocompatible matrix.

20. The method of claim 10 wherein the one or more stabilization elements are selected from sponges; self-expanding fluid; foam; pro-coagulants and glue.

21. A delivery device comprising a double lumen catheter pre-loaded with a compacted form of an endovascular device, and a stabilization element.

22. A kit for lateral stabilization of an endovascular device within an aneurysm space, the kit comprising:
a stabilization system comprising one or more stabilization elements;
a catheter suitable for delivering the stabilization system; and
instructions for use.

23. The kit according to claim 22, further comprising an imaging system for imaging the aneurysm space in conjunction with deployment of the stabilization system.

24. The kit according to claim 23, wherein the stabilization system comprises one or more balloons, and wherein the kit further comprises a biocompatible fluid suitable for filling the balloon and a catheter suitable for delivering the fluid composition to the aneurysm space.
